(19) United States
(12) Patent Application Publication
(10) Pub. No.: US 2022/0282140 A1
(43) Pub. Date: Sep. 8, 2022

(54) Photocurable thermochromic adhesive, adhesive composition and preparation as well as application thereof

(71) Applicant: Sichuan University, Chengdu (CN)

(72) Inventor: Xianglong Han, Chengdu (CN)

(21) Appl. No.: 17/824,476

(22) Filed: May 25, 2022

(30) Foreign Application Priority Data
May 25, 2021 (CN) 202110574616.X

(51) Int. Cl. C09J 175/04; C09J 11/06; A61K 6/30; C09J 5/00; C08K 5/101; C08K 5/11; C08K 5/05; C08K 5/18

(52) U.S. Cl. CPC C09J 175/04; C09J 11/06; A61K 6/30; C09J 5/00; C08K 5/101; C08K 5/11; C08K 5/05; C08K 5/18

(57) ABSTRACT

Among a photocurable thermochromic adhesive, an adhesive composition and preparation as well as application thereof, the photocurable thermochromic adhesive includes: 20-60 wt. % of acrylic polyurethane, 10-20 wt. % of Bis-GMA, 10-20 wt. % of triethylene glycol dimethacrylic acid, 10-20 wt. % of ethoxylated trimethylolpropane triacrylate, 10-20 wt. % of HEMA, 10-20 wt. % of modified silica, 1-5 wt. % of a photoinitiator component, 1-5 wt. % of BHT, 1-5 wt. % of KH570, and 1-2 wt. % of a thermochromic component. The adhesive of the present invention is colored in vitro and colorless in oral environment, which helps the doctor to easily remove residues on tooth surfaces when adhering and removing brackets.

### CROSS REFERENCE OF RELATED APPLICATION

The present invention claims priority under 35 U.S.C. 119(a-d) to CN 202110574616.X, filed May 25, 2021.

### BACKGROUND OF THE PRESENT INVENTION

### Field of Invention

The present invention relates to a technical field of adhesives, and more particularly to a technical field of photocurable thermochromic adhesives for orthodontics.

### Description of Related Arts

Orthodontics mainly studies the etiological mechanism, diagnostic analysis, prevention and treatment of malocclusion and deformity. Orthodontic treatment mainly adjusts the coordination between nerves and muscles at facial bones, teeth and maxillofacial regions through various orthodontic devices, that is, to regulate the abnormal relationship between the upper and lower jaws, between the upper and lower teeth, between the teeth and the jawbone, and between the nerves and muscles that connect them. The ultimate goal of orthodontic treatment is to achieve balance, stability and aesthetics of the oral and maxillofacial system.

The bracket adhering system used in orthodontics is usually a three-component system, which includes acid etchant, pretreatment agent and adhesive. Commercially available orthodontic adhesives are mainly photocurable materials and two-component resin materials.

In order to meet the growing demand for aesthetics, orthodontic adhesives are often designed to be close to the color of the teeth, which makes it hard for doctors to distinguish between the tooth surface and the adhesive in clinical use, causing difficulty in removing excess adhesive. Insufficient removal will cause residual adhesive on the tooth surface, and excessive removal will damage the tooth surface.

Therefore, it is of great significance to display the location and residual amount of adhesive in a reasonable manner.

Although some conventional photocurable thermochromic adhesives can also meet the requirements of discoloration, most of them cannot switch between colored and colorless. It is urgent to develop an adhesive that is capable of rapid discoloration around human oral temperatures and satisfies oral application requirements. In addition, based on the endurance requirements, good stability and effective adhering strength are very important to prolong the service life of orthodontic adhesives. However, there are few orthodontic adhesive systems in the prior art that have both good stability and sufficient adhering strength.

### SUMMARY OF THE PRESENT INVENTION

In order to overcome the above deficiencies of the prior art, an object of the present invention is to provide a photocurable thermochromic adhesive, which is colored at a lower temperature, and becomes colorless at human oral temperature. Such adhesive has sufficient adhesion, transparency, cohesion and photocuring rate, as well as good stability in oral environment.

Another object of the present invention is to provide an adhesive composition containing the photocurable thermochromic adhesive and a photocurable pretreatment agent therefor, which provides excellent adhering strength to enhance orthodontic effect, and is safe, hygienic, and convenient in oral operation.

Yet another object of the present invention is to provide preparation methods and applications of the photocurable thermochromic adhesive and the adhesive composition.

Accordingly, the present invention provides:

a photocurable thermochromic adhesive, comprising: 20-60 wt. % of acrylic polyurethane, 5-10 wt. % of bisphenol A glyceride (Bis-GMA), 10-20 wt. % of triethylene glycol dimethacrylic acid, 10-20 wt. % of ethoxylated trimethylolpropane triacrylate, 10-20 wt. % of hydroxyethyl methacrylate (HEMA), 1-2 wt. % of a photoinitiator component, 0.1-1 wt. % of butylated hydroxytoluene (BHT), and 0.01-2wt.% of a thermochromic component.

The photoinitiator component refers to a chemical substance with photo- initiating effect and/or a composition thereof The thermochromic component refers to a chemical substance and/or a composition thereof that has temperature sensitivity and can change color according to different temperatures.

In the adhesive of the present invention, cyano groups in acrylic polyurethane can provide adhesion, prepolymer can provide cohesion and good morphology to the product, and long chain can provide flexibility. Due to a biphenyl ring structure, Bis-GMA can provide good rigidity. Triethylene glycol dimethacrylic acid has good fluidity, which can reduce viscosity of a resin system, thus greatly affecting an adding amount of the filler, while aliphatic chain of triethylene glycol dimethacrylic acid can provide toughness. In addition to providing fluidity, ethoxylated trimethylolpropane triacrylate can also synergize with other ingredients to provide a higher degree of crosslinking due to its trifunctional characteristics. In addition to providing fluidity, HEMA can also make the product more viscous and reduce a contact angle with a tooth surface. The photoinitiator component not only provides a proper photo-initiating ability, but also, if dosage is appropriate, cooperates with other ingredients, so as to maintain sufficient transparency and optimize a curing rate. An appropriate amount of BHT can cooperate with other components to stabilize polymerization, thereby achieving ideal mechanical and optical properties of final products. The thermochromic component not only provides thermochromic effects, but also, with appropriate amount and selection, synergizes with other components to eliminate stress concentration of the product, which makes the product evenly stressed while improves biosafety. With appropriate amounts, the above components can synergize with each other, which not only ensures good toughness, stiffness and adhering strength, but also provides good fluidity and bonding force with the tooth surface besides good biocompatibility and force uniformity.

Preferably, a molecular weight of the acrylic polyurethane is 1000-10000, and/or a viscosity thereof is 10000-30000 cps.

Preferably, the thermochromic component is selected from a group consisting of a thermochromic dye and/or a composite thereof, a polythiophene thermochromic pigment, and a liquid crystal derivative of cholesterol.

Preferably, the composite of the thermochromic dye consists of a pH-sensitive dye, a color developer and a non-volatile hydrophobic solvent.

Preferably, the pH-sensitive dye is selected from a group consisting of spironolactone compounds such as diarylphthalate, epi-cryptoacetalide, and fluorane.

Preferably, the color developer is selected from a group consisting of proton-donating compounds such as carboxylic acid compounds, phenolic compounds, and Lewis acid compounds, for example, phenol.

Preferably, the non-volatile hydrophobic solvent is selected from a group consisting of fatty acids with a melting point of 30-37° C., amides and alcohols, for example, hexadecanol.

Preferably, the liquid crystal derivative of cholesterol is selected from a group consisting of cholesterol pelargonate, cholesterol oleyl carbonate, cholesterol acetate, cholesterol benzoate and cholesterol chloride.

Preferably, the photoinitiator component comprises a free-radical photoinitiator.

Preferably, the photoinitiator component is camphorquinone and/or a hydrogen-abstracting photoinitiator.

Preferably, the hydrogen-abstracting photoinitiator is 2-ethylhexyl 4-(dimethylamino)benzoate.

Preferably, the photocurable thermochromic adhesive further comprises a filler selected from a group consisting of silica, ground glass powder containing heavy metal dust, and prepolymer.

Preferably, the filler is silica with a particle size of 0.015-10 μm.

The present invention also provides a method for preparing the photocurable thermochromic adhesive, comprising steps of:

(1) mixing the acrylic polyurethane, the Bis-GMA, the triethylene glycol to dimethacrylic acid, the ethoxylated trimethylolpropane triacrylate, and the HEMA according to a certain proportion;

(2) adding the BHT and the photoinitiator component to a mixture obtained in the step (1); and

(3) adding fumed silica and the thermochromic component to a mixture obtained in the step (2), and stirring to reach a plasticine state which is homogeneous and viscous, thereby obtaining the photocurable thermochromic adhesive.

Preferably, the thermochromic component is formed by the thermochromic dye and/or the composite thereof and/or the liquid crystal derivative of cholesterol. Before adding, the thermochromic component is made into a microcapsule composition.

The present invention also provides an adhesive composition, comprising the above photocurable thermochromic adhesive and/or a photocurable thermochromic adhesive prepared by the above method, and a photocuring pretreatment agent.

Preferably, the photocuring pretreatment agent comprises 10-20% of bisphenol A glyceride, 20-80% of hydroxyethyl methacrylate, 0.1-2% of the photoinitiator component, 1-5 wt. % of 3-(methacryloyloxy)propyltrimethoxysilane, and 0.1-5% of ethanol.

Preferably, the photocuring pretreatment agent further comprises a filler.

Preferably, in the adhesive composition, the photocurable thermochromic adhesive comprises: 20 wt. % of acrylic polyurethane, 5.6 wt. % of bisphenol A glyceride (Bis-GMA), 12 wt. % of triethylene glycol dimethacrylic acid, 6.5 wt. % of ethoxylated trimethylolpropane triacrylate, 18.6 wt. % of hydroxyethyl methacrylate (HEMA), 0.3 wt. % of camphorquinone, 1.6 wt. % of 2-ethylhexyl 4-(dimethylamino)benzoate, 0.1 wt. % of butylated hydroxytoluene (BHT), and 0.04 wt. % of the thermochromic component; the photocuring pretreatment agent comprises:

18.7% of bisphenol A glyceride, 35.6% of hydroxyethyl methacrylate, 0.64 wt. % of camphorquinone, 1.32 wt. % of 2-ethylhexyl 4-(dimethylamino)benzoate, 5 wt. % of 3-(methacryloyloxy)propyltrimethoxysilane, and 3.7% of ethanol.

Preferably, a method for preparing the photocuring pretreatment agent comprises steps of:

(1) mixing the bisphenol A glyceride, the hydroxyethyl methacrylate, the 3-(methacryloyloxy)propyltrimethoxysilane and the ethanol according to a certain proportion, and thoroughly stirring; and

(2) adding the photoinitiator component to a mixture obtained in the step (1), and thoroughly stirring while avoiding light, so as to obtain the photocuring pretreatment agent.

The present invention also provides a method for performing orthodontics, comprising a step of using the photocurable thermochromic adhesive and/or the adhesive composition.

Preferably, the method for performing the orthodontics comprises specific steps of: applying the adhesive to a metal bracket for orthodontics, then applying the pretreatment agent evenly on the tooth surfaces, and then placing the bracket on the teeth to press the adhesive against the tooth surfaces; removing excessive adhesive, and irradiating the bracket for 10-40s with a medical ultraviolet curing lamp, to obtain a cured bracket sample.

The present invention has the following beneficial effects:

The adhesive of the present invention can rapidly change color at 33° C., namely being colorless when the temperature is higher than 33° C., and colored when the temperature is lower than 33° C.

The adhesive of the present invention is colored at a lower temperature, and is colorless at a human oral temperature, so it will not affect the appearance in the daily orthodontic process. When it needs to be removed, the adhesive can be colored by cooling, facilitating removal.

The adhesive composition of the present invention can be rapidly cured under the irradiation of an LED ultraviolet light, and can reach a shear strength of about 3 MPa in about 20 s, which is enough to fix the bracket on the tooth surfaces. Furthermore, a short curing time can save operating time for doctors.

The shear strength of the adhesive composition of the present invention is no less than 7 MPa, which meets the strength requirements of orthodontic repair and reduces the falling probability of the bracket.

The adhesive composition of the present invention can stably maintain an excellent adhering strength in the oral cavity of a human body for a long period of time.

### BRIEF DESCRIPTION OF THE DRAWINGS

FIG. 1 illustrates a shear test device used in embodiment 2 of the present invention;

FIG. 2 is a comparison of a sample 1 obtained in embodiment 3 of the present invention before and after discoloration;

FIG. 3 is a comparison of a sample 2 obtained in the embodiment 3 of the present invention before and after discoloration;

FIG. 4 is a comparison of a sample 3 obtained in the embodiment 3 of the present invention before and after discoloration;

FIG. 5 is a comparison of a sample 4 obtained in the embodiment 3 of the present invention before and after discoloration; and

FIG. 6 is a shear strength stability test curve according to embodiment 23 of the present invention.

### DETAILED DESCRIPTION OF THE PREFERRED EMBODIMENT

Referring to drawings and embodiments, the present invention will be further illustrated. However, the embodiments and drawings are exemplary only and not intended to be limiting. All reasonable modifications and combinations made within the inventive concept of the present invention shall fall into the protection scope of the present invention.

### EMBODIMENT 1

A thermochromic component is prepared by:

1. Thermochromic dyes and/or composite thereof

(1) mixing thermochromic green (CAS: 29512-46-7) or thermochromic blue (87563-89-1), phenol and hexadecanol at a mass ratio=1:1:100, and then heating until the system is melted; thoroughly mixing the system in a melted state; and

(2) cooling a mixture obtained in the step (1) to room temperature, and preparing into microcapsules to obtain dark green or dark blue powder.

2. Liquid crystal derivative of cholesterol

(1) mixing cholesterol pelargonate, cholesterol oleyl carbonate, cholesterol acetate and cholesterol benzoate according to a certain mass ratio, and preparing into microcapsules to obtain green powder.

### EMBODIMENT 2

A photocurable thermochromic adhesive for orthodontics is prepared by:

(1) mixing acrylic polyurethane with a molecular weight of about 1700, Bis-GMA (bisphenol A glyceride), triethylene glycol dimethacrylic acid (CAS: 109-16-0), ethoxylated trimethylolpropane triacrylate (CAS: 28961-43-5), and HEMA (hydroxyethyl methacrylate) according to a certain proportion, until the whole system is fully mixed;

(2) adding BHT (antioxidant 264) and a photoinitiator component consisting of camphorquinone and 2-ethylhexyl 4-(dimethylamino)benzoate (mass ratio of 1:1.5) into a mixture obtained in the step (1), and thoroughly stirring; and

(3) adding a filler and the thermochromic component obtained in the embodiment 1 to a mixture obtained in the step (2), and stirring to reach a plasticine state which is homogeneous and viscous, thereby obtaining the adhesive which can be completely cross-linked and cured by curing for 20 s with a medical UV curing light;

wherein a specific mass ratio of each raw material is: acrylic polyurethane 35 wt. %, Bis-GMA 5 wt. %, triethylene glycol dimethacrylate acid 10 wt. %, ethoxylated trimethylolpropane triacrylate 20 wt. %, HEMA 10 wt. % %, photoinitiator system 2wt. %, BHT 1 wt. %, thermochromic component 1 wt. %, and the balance of fumed silica.

A photocurable pretreatment agent is prepared by:

(1) mixing bisphenol A glyceride, hydroxyethyl methacrylate, 3-(methacryloyloxy)propyltrimethoxysilane and ethanol according to a certain proportion and thoroughly stirring; and

(2) add the same photoinitiator component used in preparation of the photocurable thermochromic adhesive to a mixture obtained in the step (1), thoroughly stirring and avoiding light;

wherein a specific mass ratio of each raw material is: bisphenol A glyceride 15%, hydroxyethyl methacrylate 75%, photoinitiator component 1%, 3-(methacryloyloxy)propyltrimethoxysilane 4%, and ethanol 5%.

The final photocurable thermochromic adhesive is a solid adhesive in the plasticine state, which is thermochromic. The photocurable pretreatment agent is a viscous transparent yellow liquid.

### EMBODIMENT 3

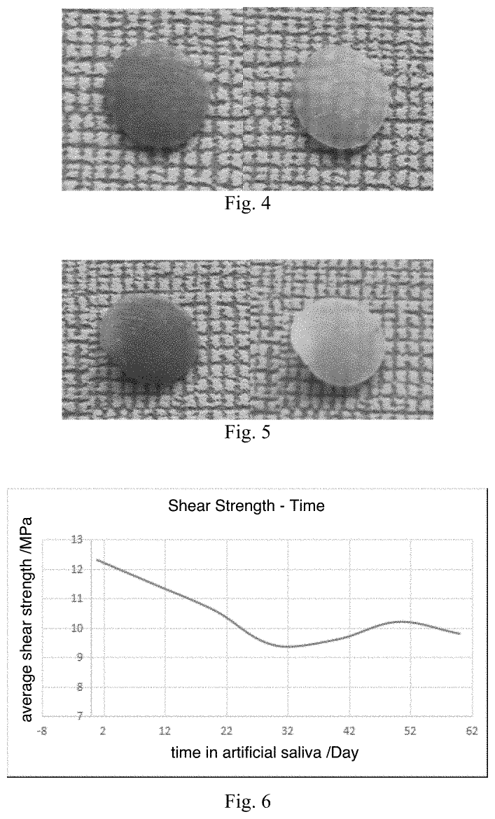

Another thermochromic component is prepared according to the process of the embodiment 1, and then the photocurable thermochromic adhesive for orthodontics is prepared according to the process of the embodiment 2, to obtain adhesives shown in FIGS. 2-5. According to color tests, sample 1 showed dark green (C100, Y100) below 22° C., and the color faded and became nearly colorless above 22° C.; sample 2 was dark blue below 22° C. (C100, M100), and the color faded and became nearly colorless above 22° C.; sample 3 showed peacock green (C60, Y25) below 33° C., and the color faded and became nearly colorless above 33° C.; sample 4 showed bright blue (C100, M60) below 33 showed, and the color faded and became nearly colorless above 33° C.

### EMBODIMENTS 4-6

The adhesive is prepared by the method of the embodiment 2, wherein types of the thermochromic component are changed, while other components remain. Results are shown in the following table:

TABLE 1
test results of thermochromic effect according to the embodiments 4-6

| | Types of thermochromic component | | | |
|---|---|---|---|---|
| | | | Thermochromic dyes | |
| | Thermochromic | | and/or composite | |
| | dyes and/or | Liquid crystal | thereof and liquid | |
| | composite | derivative of | crystal derivative of | Thermochromic |
| Embodiments | thereof | cholesterol | cholesterol | effect |
| 4 | yes | no | no | Rapid (a few seconds), |
| | | | | repeatable |
| 5 | no | yes | no | Slow (tens of seconds), |
| | | | | repeatable |
| 6 | no | no | yes | Normal (over ten |
| | | | | seconds), repeatable |

### EMBODIMENT 7

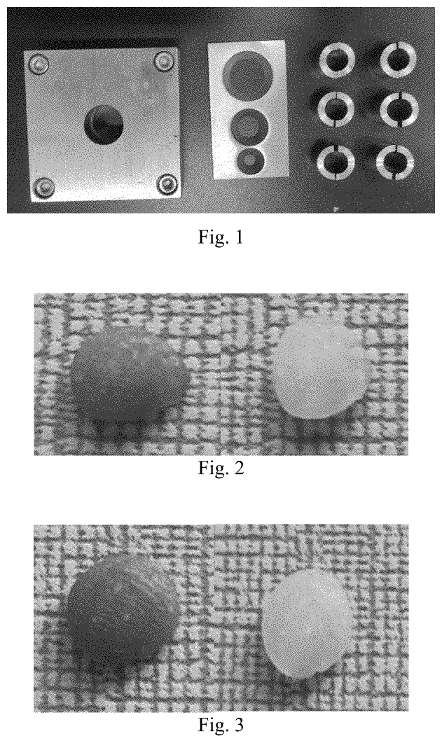

Shear strength of the adhesive composition obtained in the embodiment 2 is tested by a device shown in FIG. 1 and the following processes:

taking anterior teeth from bovine teeth, excise tooth roots under running water with an emery sheet, and cutting into a suitable size that fits a dental cup; then embedding mesial, distal and palatal surfaces of a dental crown with super anhydrite or self-curing resin in the dental cup; exposing a buccal surface of the dental crown to be slightly above a rim of the dental cup;

grinding the embedded teeth on a grinder with sandpapers from low to high meshes in turn, until tooth surfaces are smooth; and

cleaning the tooth surfaces with alcohol and drying with oil-free gas;

evenly applying an acid etchant on the tooth surfaces, etching for 20-30s, then rinsing off the acid etchant with high-speed running water, and then drying the tooth surfaces with high-pressure oil-free gas;

evenly applying the adhesive prepared in the embodiment 1 on a orthodontic flat-bottom metal bracket, and then evenly applying the prepared pretreatment agent on the tooth surfaces; placing the bracket on the tooth surfaces and pressing gently so that the adhesive is pressed evenly, and then removing excess adhesive; and

irradiating with a medical UV curing light for 20s to complete installation of a bracket sample.

Multiple samples prepared according to the above processes are tested as follows, and specific test processes are based on 6.5 Adhering Strength Test Method in YY/T 0269-2009 Orthodontic Bracket adhering Materials. Test results of shear adhering strength are as follows:

TABLE 2
sample shear strength of the embodiment 7

| Sample number | 1 | 2 | 3 | 4 | 5 | 6 |
|---|---|---|---|---|---|---|
| Shear strength/MPa | 4.7 | 7.3 | 6.7 | 4.6 | 3.6 | 7.9 |

| | |
|---|---|
| Average ± error/MPa | 5.80 ± 1.58 |

It can be seen that the adhesive strength for orthodontics prepared in the embodiment 7 is 3.6-7.9 MPa after photocuring for 20s.

### EMBODIMENTS 8-14

Photocurable thermochromic adhesives of the embodiments 8-14 for orthodontics are prepared by:

(1) mixing acrylic polyurethane with a viscosity of 15000-20000 cps, Bis-GMA, triethylene glycol dimethacrylic acid, ethoxylated trimethylolpropane triacrylate, and HEMA according to a certain proportion, until the whole system is fully mixed;

(2) adding BHT and a photoinitiator component into a mixture obtained in the step (1), and thoroughly stirring; and (3) adding a filler and the thermochromic component obtained in the embodiment 1 with the thermochromic dyes and/or the composite thereof to a mixture obtained in the step (2), and stirring to reach a plasticine state which is homogeneous and viscous, thereby obtaining the adhesive which can be completely cross-linked and cured by curing for 20s with a medical UV curing light;

wherein the photocurable pretreatment agent adopts the formula of the embodiment 2. Raw material proportions of the photocurable thermochromic adhesives of each embodiment are shown in Table 3:

TABLE 3
raw material proportions of the embodiment 8-14

| | Embodiment | | | | | | |
|---|---|---|---|---|---|---|---|
| Component (wt. %) | 8 | 9 | 10 | 11 | 12 | 13 | 14 |
| acrylic polyurethane | 20 | 32 | 43.5 | 36.9 | 53.6 | 60 | 47.8 |
| (15000-20000 cps) | | | | | | | |
| Bis-GMA | 5.6 | 7.8 | 9.3 | 6.5 | 5 | 10 | 8.3 |
| triethylene glycol | 12 | 12.1 | 13.5 | 13.6 | 18.6 | 19 | 10.2 |
| dimethacrylate acid | | | | | | | |
| ethoxylated | 16.5 | 15.9 | 15.6 | 12 | 17.6 | 19.7 | 12 |
| trimethylolpropane | | | | | | | |
| triacrylate | | | | | | | |
| HEMA | 18.6 | 14.4 | 10 | 15.4 | 20.4 | 18.5 | 17.5 |
| fumed silica | balance | | | | | | |
| camphorquinone | 0.3 | 1 | 0.5 | 0.78 | 1 | 0.8 | 1.5 |
| 2-ethylhexyl 4- | 1.6 | 1.5 | 1 | 0.45 | 1 | 1.2 | 0.5 |
| (dimethylamino) | | | | | | | |
| benzoate | | | | | | | |
| BHT | 0.1 | 0.3 | 0.56 | 0.49 | 1 | 0.78 | 0.36 |
| thermochromic | 0.04 | 1.36 | 0.83 | 1 | 0.1 | 1.86 | 0.65 |
| component | | | | | | | |

### EMBODIMENTS 15-21

Pretreatment agents are prepared according to the method of the embodiment 2:

TABLE 4
raw material proportions of the embodiment 15-21

| | Embodiment | | | | | | |
|---|---|---|---|---|---|---|---|
| Component (wt. %) | 15 | 16 | 17 | 18 | 19 | 20 | 21 |
| bisphenol A | 11.3 | 15.6 | 16.9 | 18.7 | 13.2 | 17.6 | 14.5 |
| glyceride | | | | | | | |
| ethoxylated | 56.3 | 70.9 | 50.3 | 35.6 | 49.6 | 66.5 | 40.8 |
| trimethylolpropane | | | | | | | |
| triacrylate | | | | | | | |
| camphorquinone | 0.3 | 0.53 | 0.1 | 0.64 | 0.92 | 0.82 | 0.45 |
| 2-ethylhexyl 4- | 0.6 | 1.46 | 0.3 | 1.32 | 1.03 | 0.64 | 0.53 |
| (dimethylamino) | | | | | | | |
| benzoate | | | | | | | |
| 3- | 1.3 | 2.6 | 3.7 | 5 | 4.6 | 3.5 | 2.6 |
| (methacryloyloxy) | | | | | | | |
| propyltrimethoxysilane | | | | | | | |
| ethanol | 0.5 | 1.3 | 2.1 | 3.7 | 5 | 0.9 | 1.7 |

| | |
|---|---|
| fumed silica | balance |

5

### EMBODIMENT 22

According to the method of the embodiment 7, the adhesive is used together with the pretreatment agent to perform shear strength tests. Results are shown in the following table:

TABLE 5
shear strength test results

| Embodiment | | | | | | | |
|---|---|---|---|---|---|---|---|
| Strength | 8 | 9 | 10 | 11 | 12 | 13 | 14 |
| 15 | 3.8 | 4.8 | 3.2 | 3 | 6.7 | 7.3 | 4.7 |
| 16 | 6 | 2.6 | 7.1 | 5 | 11 | 4.2 | 4.5 |
| 17 | 2.1 | 6.3 | 9.6 | 4.3 | 8.7 | 7.5 | 11.7 |
| 18 | 12.3 | 8.6 | 4.5 | 6.7 | 2.7 | 2.1 | 2.7 |
| 19 | 3.4 | 2.5 | 4.1 | 3.4 | 6.2 | 5.8 | 9.1 |
| 20 | 8.1 | 6.6 | 9.7 | 11.3 | 5.3 | 5.7 | 4.3 |
| 21 | 3.9 | 3.7 | 1.7 | 8.5 | 10.6 | 6.4 | 9.2 |

It can be seen from the table that the combination of the embodiments 18 and 8 can optimize the strength.

### Embodiment 23

50 samples of the optimal combination according to the embodiment 22 were put into artificial saliva, and 10 samples were taken out every 10 days to carry out the shear strength test according to the method of the embodiment 7. Results are shown in FIG. 6.

It can be seen from FIG. 6 that the shear strength of the optimal combination in the embodiment 22 is finally stabled at about 10 MPa, which meets the clinical strength requirements of orthodontic repair.

The above embodiments are only preferred embodiments of the present invention, and the protection scope of the present invention is not limited thereby. All technical solutions under the concept of the present invention fall into the protection scope of the present invention. It should be pointed out that for those skilled in the art, improvements and modifications without departing from the principles of the present invention should also be regarded as covered by the protection scope of the present invention.

What is claimed is:

1. A photocurable thermochromic adhesive, comprising: 20-60 wt. % of acrylic polyurethane, 5-10 wt. % of bisphenol A glyceride, 10-20 wt. % of triethylene glycol dimethacrylic acid, 10-20 wt. % of ethoxylated trimethylolpropane triacrylate, 10-20 wt. % of hydroxyethyl methacrylate, 1-2 wt. % of a photoinitiator component, 0.1-1 wt. % of dibutylhydroxytoluene, and 0.01-2 wt. % of a thermochromic component.

2. The photocurable thermochromic adhesive, as recited in claim 1, wherein the thermochromic component is selected from a group consisting of a thermochromic dye and/or a composite thereof, a polythiophene thermochromic pigment, and a liquid crystal derivative of cholesterol.

3. The photocurable thermochromic adhesive, as recited in claim 2, wherein the composite of the thermochromic dye consists of a pH-sensitive dye, a color developer and a non-volatile hydrophobic solvent, wherein the color developer is selected from a group consisting of proton-donating compounds; the pH-sensitive dye is selected from a group consisting of spironolactone compounds; and the non-volatile hydrophobic solvent is selected from a group consisting of fatty acids with a melting point of 30-37° C., amides and alcohols.

4. The photocurable thermochromic adhesive, as recited in claim 1, wherein the photoinitiator component is camphorquinone and/or a hydrogen-abstracting photoinitiator.

5. The photocurable thermochromic adhesive, as recited in claim 4, wherein the hydrogen-abstracting photoinitiator is 2-ethylhexyl 4-(dimethylamino)benzoate.

6. The photocurable thermochromic adhesive, as recited in claim 1, further comprising a filler selected from a group consisting of silica, ground glass powder containing heavy metal dust, and prepolymer.

7. A method for preparing the photocurable thermochromic adhesive as recited in claim 6, comprising steps of:
(1) mixing the acrylic polyurethane, the bisphenol A glyceride, the triethylene glycol dimethacrylic acid, the ethoxylated trimethylolpropane triacrylate, the hydroxyethyl methacrylate and 3-(methacryloyloxy)propyltrimethoxysilane according to a certain proportion;
(2) adding the dibutylhydroxytoluene and the photoinitiator component to a mixture obtained in the step (1); and
(3) adding the filler and the thermochromic component to a mixture obtained in the step (2), and stirring to reach a plasticine state which is homogeneous and viscous, thereby obtaining the photocurable thermochromic adhesive.

8. The method, as recited in claim 7, wherein before adding, the thermochromic component is made into a microcapsule composition.

9. An adhesive composition, comprising the photocurable thermochromic adhesive as recited in claim 6 and a photocuring pretreatment agent, wherein the photocuring pretreatment agent comprises 10-20% of bisphenol A glyceride, 20-80% of hydroxyethyl methacrylate, 0.1-2% of the photoinitiator component, 1-5wt. % of 3-(methacryloyloxy)propyltrimethoxysilane, and 0.1-5% of ethanol.

10. An adhesive composition, comprising the photocurable thermochromic adhesive prepared by the method as recited in claim 8 and a photocuring pretreatment agent, wherein the photocuring pretreatment agent comprises 10-20% of bisphenol A glyceride, 20-80% of hydroxyethyl methacrylate, 0.1-2% of the photoinitiator component, 1-5 wt. % of 3-(methacryloyloxy)propyltrimethoxysilane, and 0.1-5% of ethanol.

11. A method for performing orthodontics, comprising a step of using the photocurable thermochromic adhesive as recited in claim 6.

12. A method for performing orthodontics, comprising a step of using the adhesive composition as recited in claim 9.
